(19) United States
(12) Patent Application Publication
(10) Pub. No.: US 2026/0004923 A1
(43) Pub. Date: Jan. 1, 2026

(54) System and Method for Managing the Generation of Corrective Action Plans

(71) Applicant: Matthew Just, Duluth, MN (US)

(72) Inventor: Matthew Just, Duluth, MN (US)

(21) Appl. No.: 19/319,911

(22) Filed: Sep. 5, 2025

Related U.S. Application Data
(63) Continuation-in-part of application No. 18/616,474, filed on Mar. 26, 2024.

(51) Int. Cl. G16H 40/20; G06F 40/30

(52) U.S. Cl. CPC G16H 40/20; G06F 40/30

(57) ABSTRACT

A system and method for managing the generation of corrective action plans is described in which a listing of required corrective actions—including a recital of the factual basis underlying each requirement—is provided, a large language model identifies the recital, and a corrective action plan is formed based upon the recital. A computer interface permits uploading the listing and generating, editing, saving, and exporting the corrective action plan. The invention provides technical improvements by converting unstructured regulatory text into a schema-normalized structured representation with indexed retrieval and deterministic assembly, reducing I/O operations and recomputation and thereby improving computer system efficiency.

### RELATED APPLICATIONS

This application is a continuation-in-part of, and relates and claims priority to, U.S. patent application Ser. No. 18/616,474, filed on Mar. 26, 2024, now pending, hereby incorporated by reference herein for all purposes.

### FIELD OF THE DISCLOSURE

The invention relates generally in one embodiment to a system and method for managing the generation of corrective action plans, and more particularly, to the use of a large language model in the generation of corrective action plans.

### BACKGROUND

### 1. Large Language Models

Large language models are advanced artificial intelligence systems designed to understand and generate human-like text. These models are trained on vast amounts of text data from the internet and other sources, allowing them to learn the patterns and nuances of human language.

Large language models work by using deep learning techniques to process and analyze text data. They consist of multiple layers of artificial neural networks that operate to understand the context of a given input and generate relevant and coherent text based on that context.

Transformer-based large language models (LLMs) are one example of such models and may be used to generate contextually relevant text across a range of inputs. The disclosure is not limited to any particular vendor or model version.

Large language models have a wide range of applications, including natural language understanding, text generation, translation, summarization, and more. They are used in various industries such as customer service, content generation, language translation, and academic research, among others.

### 2. Regulatory Audits of Health Care Facilities

Health care facilities must comply with numerous state and federal regulations. For example, a health care provider or supplier would be reviewed by the Department of Health and Human Services Centers for Medicare and Medicaid Services (“CMS”) in connection with a certification or recertification process. The health care facility would be visited by certification personnel to ensure that the facility meets the various applicable requirements set forth in the Code of Federal Regulations. Typically, the health care facility receives a notice of the results of such an audit. Where shortcomings exist, the notice typically includes a listing of the regulation that is not met and specific details of the audit's findings as to why the particular regulation is not met. The health care facility is then afforded a specified amount of time to present to the auditors a plan of corrective action to bring the health care facility into compliance with the particular regulation at issue.

There are hundreds if not thousands of regulations that a health care facility must meet. Thus, an audit notice might include numerous regulations that are not met, and for each of which a plan of corrective action is required. Responding to an audit notice accordingly would be an extremely difficult, expensive, and time-consuming process.

For convenience only, reference is made herein to CMS audit notices, but the invention is not so limited. A person of ordinary skill in the art having the benefit of this disclosure would appreciate the application of the invention in other contexts, e.g., where plans of corrective action need to be developed.

### SUMMARY

The present disclosure in one embodiment provides a system and method for managing the generation of corrective actions plans. In one embodiment, a listing of corrective actions required is provided. The listing in one embodiment may include a listing of regulations for which corrective action is required, and a recital of the factual basis underlying each requirement for corrective action. A large language model may be used to identify the recital in the listing, and then form a corrective action plan based upon such recital. Alternately, the listing may be parsed and the recital provided to the large language model. A computer interface is provided through which the listing, or a portion thereof, may be uploaded and the corrective action plan generated. The computer interface may also permit for the editing of the generated corrective action plan. The computer interface may permit the printing of a generated or edited corrective action plan. The computer interface may also permit the saving to memory of a generated or edited corrective action plan. The computer interface may also classify a generated or edited corrective action plan as being a work in progress (i.e., in review) or a work in final form.

In certain embodiments, the system first converts regulatory findings into a structured representation (e.g., JSON conforming to a predefined schema), maps each deficiency to a canonical rule identifier, and then assembles a compliant corrective action plan using a deterministic state-machine pipeline with automated completeness checks. By operating over the structured representation rather than repeatedly parsing source documents, the system reduces CPU cycles and disk I/O, enables indexed lookups for rule constraints, and supports caching and reuse of validated plan components.

Other benefits and advantages of the present disclosure will be appreciated from the following detailed description.

### DESCRIPTION OF THE DRAWINGS

FIG. 1 is a block diagram illustrating one exemplary embodiment of a server system that may be used to implement certain systems and methods described herein.

FIG. 2 is a block diagram illustrating one exemplary embodiment of a serverless hosting system that may be used to implement certain systems and methods described herein.

### DETAILED DESCRIPTION

Embodiments of the invention and various alternatives are described. Those skilled in the art will recognize, given the teachings herein, that numerous alternatives and equivalents exist which do not depart from the invention. It is therefore intended that the invention not be limited by the description set forth herein or below.

One or more specific embodiments of the system and method will be described below. These described embodiments are only exemplary of the present disclosure. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, for clarity and convenience only, and without limitation, the disclosure (including the drawings) sets forth exemplary representations of only certain aspects of events and/or circumstances related to this disclosure. Those skilled in the art will recognize, given the teachings herein, additional such aspects, events and/or circumstances related to this disclosure, e.g., additional elements of the devices described; events occurring related to the creation of corrective action plans; etc. Such aspects related to this disclosure do not depart from the invention, and it is therefore intended that the invention not be limited by the certain aspects set forth of the events and circumstances related to this disclosure.

As used herein, “recital” refers to the factual narrative of surveyor or auditor findings underlying a cited regulatory deficiency, including the specific observations, dates, locations, and circumstances documented by the auditor.

A server may include a database having information in the form of a listing of corrective actions required for a particular environment. In one embodiment, the environment may be a healthcare facility, and the listing may originate from the CMS.

A user computing device or controller may be used to populate the database with listings for one or more environments. Related data files may be linked by a common entry identification.

The user computing device or controller may be used to access a data entry interface. The data entry interface may have the ability to receive data for transfer to the server. The data entry interface may take any of a number of forms, e.g., radio buttons, drop-down menus, text entry fields, etc.

When entering information about a new or potential listing, staff may be prompted to enter information related to the listing. In that way, data files are created which may be accessed later for modification or use.

Upon entry of a data file, a large language model may be used to identify in a corrective action listing a recital of the factual basis underlying each requirement for corrective action. The user computing device or controller may be used to access a user interface that provides an option to initiate the recital identification process. The user interface may take any of a number of different forms, e.g., radio buttons, drop-down menus, text entry fields, etc. In one embodiment, the large language model may identify in a corrective action listing a heading or other indicator of the location of the recital. Alternately, the listing may be parsed and the recital provided to the large language model.

Once the recital identification process is complete, the large language model may be used to generate a corrective action plan. The corrective action plan may include a specific corrective action for each requirement for corrective action present in the identified recital. The corrective action plan may be saved to the database in a data file.

The user computing device or controller may be used to access the database and retrieve all or a portion of a data file. An interface is provided to display any retrieved information. The interface in one embodiment may display all or a portion of a corrective action plan. The interface may permit editing of the corrective action plan, either manually or by further action of the large language model. The interface may permit any version of the corrective action plan to be saved to the database. The interface also may allow a status indicator to be assigned to the corrective action plan. The status indicator may be set to correspond to the current state of the corrective action plan (e.g., new, modified, under review, under final review, final, etc.).

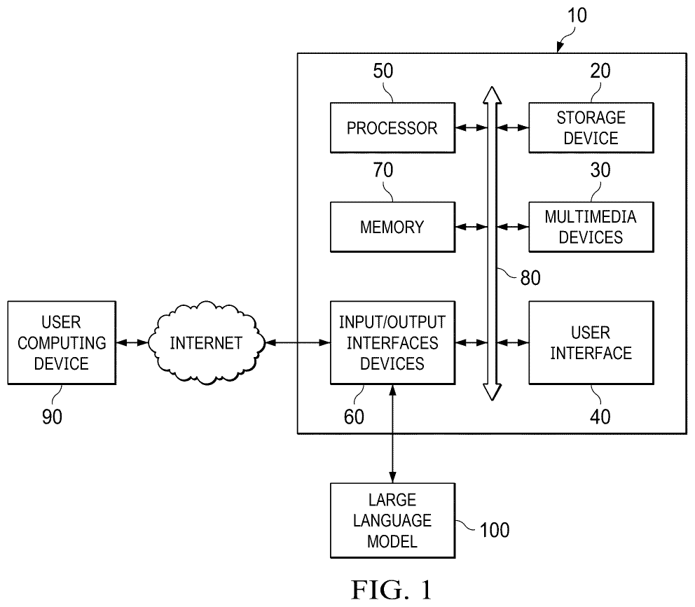

As shown in FIG. 1, the server 10 may comprise one or more servers and/or personal computers. The server may include a memory 70, which may include a random-access memory for temporary information storage, a storage device 20 including a read-only memory for permanent information storage, a hard drive, diskette, optical media storage device, flash drive, etc. The server 10 typically may include a standards-based bus system 80 for communication between various server modules, e.g., input/output interfaces or devices 60, display devices 40, multimedia devices 30, memory 70, mass-storage devices 20, processing units 50, etc.

The server 10 may communicate via the internet with a user computing device 90, e.g., a personal computer, a tablet, a smartphone, etc. The server 10 also communicates with large language model 100 for the generation of a corrective action plan.

In one embodiment, a server computing system for providing corrective action plan information to a user is provided, the server computing system comprising:
a database storing corrective-action-related information for a healthcare facility;
a non-transitory computer readable storage device configured to store software instructions;
a computer processor configured to execute the software instructions to:
   serve a website or mobile application content including user interface content renderable on a user computing device, wherein the user interface content comprises dynamic user interface controls for receiving information for the healthcare facility and presenting for the healthcare facility corrective-action-related information;
   communicate the website or mobile application content to the user computing device, wherein the user computing device renders the website or mobile application content to display the user interface content;
   receive the corrective-action-related information for the healthcare facility, wherein the corrective-action-related information is inputted via the dynamic user interface controls on the user computing device;
   access from the database a recital of corrective action requirements;
   serve an updated website or mobile application content including updated user interface content including a corrective action plan generated at least in part by a large language model; and
   communicate the updated website or mobile application content to the user computing device, wherein the updated website or mobile application content is renderable by the user computing device to display the updated user interface content.

In an alternate embodiment, a cloud-native application is provided in a serverless hosting environment. The cloud-native application is designed and built to leverage cloud computing principles and take full advantage of cloud platform capabilities. The application may be designed with a microservices architecture, where individual components or services are loosely coupled and independently deployable. The cloud-native application may be containerized, meaning that it is packaged with all its dependencies and can run consistently across different environments.

Serverless hosting refers to a cloud computing model where a cloud provider dynamically manages the allocation and provisioning of servers to host applications. In this model, developers can focus on writing and deploying code without needing to manage or provision servers themselves. Serverless hosting platforms, such as AWS Lambda, Azure Functions, and Google Cloud Functions, execute code in response to events or requests, automatically scaling up or down based on demand.

The phrase “serve a website” means to make the files and resources of a website (like HTML pages, CSS stylesheets, JavaScript, images, etc.) available to users over the internet (or a network) through a web server. Here's how it works in practice:
1. Hosting files-The website's content (HTML, images, scripts) is stored on a computer (the server).
2. Listening for requests—The server runs software (like Apache, Nginx, or Node.js) that waits for incoming requests, usually via the HTTP or HTTPS protocol.
3. Responding to requests—When a user types a URL into their browser, the browser sends an HTTP request to the server.
4. Delivering content—The server finds the requested file (or generates it dynamically) and sends it back to the browser.
5. Rendering—The browser takes those files and displays the website to the user.

So in short: serving a website equals delivering web content from a server to a client (browser) upon request.

The phrase “serve mobile application content” means:
1. Backend provides data—A server hosts content (text, images, video, user data, etc.) that the mobile app needs.
2. App requests content—The app makes requests over the internet (usually through an API, often REST or GraphQL).
3. Server responds—The backend sends the requested content back, often in JSON or XML format, or as media files.
4. App displays it—The mobile app renders that data into a user-friendly interface.

Here are some examples:
A. A news app doesn't store all articles inside the app itself. Instead, it requests the latest articles from a server, which serves that content to the app.
B. A music app like Spotify streams songs from servers—those servers are “serving” the audio content to a phone.

As compared to serving a website, for websites, servers deliver HTML/CSS/JS to a browser. For mobile apps, servers deliver raw data or media (not a whole webpage), and the app itself handles how it looks.

These two definitions are provided for clarity and do not limit the scope of the claims.

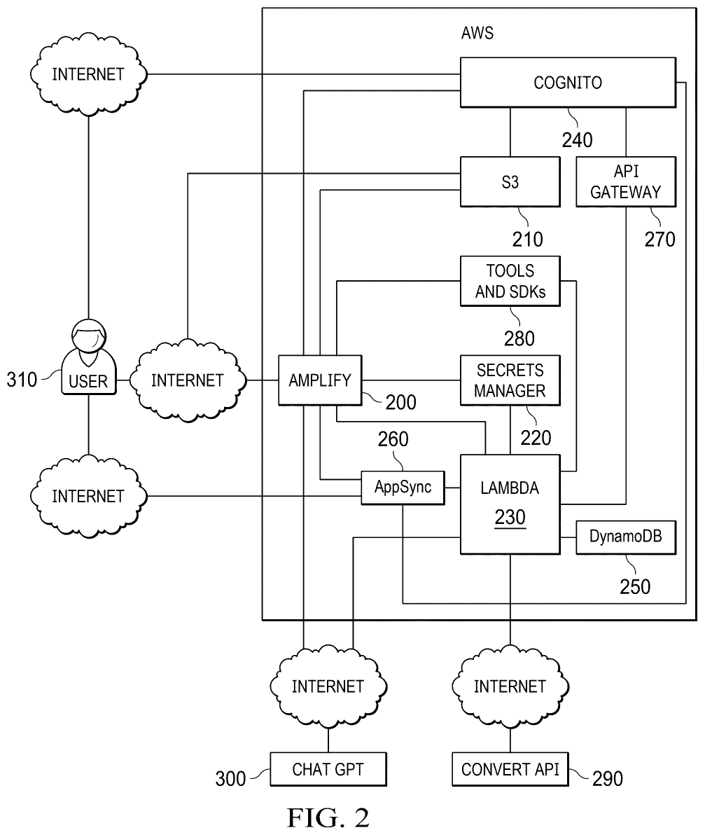

Shown in FIG. 2 is an exemplary Amazon Web Services (AWS) serverless hosting platform accessible by a user 310. As described in FIG. 2:
“Amplify” 200 is a set of tools and services designed to help developers build full-stack web and mobile applications quickly and easily. It provides features such as hosting, authentication, GraphQL APIs, analytics, and more, all with a focus on simplifying the development process and improving scalability and performance.
“S3” 210 refers to Amazon Simple Storage Service, a scalable cloud storage service provided by AWS. It allows users to store and retrieve data from anywhere on the web, making it highly flexible and accessible. S3 is commonly used for storing a wide variety of data types, including images, videos, backups, and application data. It offers features such as high availability, durability, security, and scalability, making it a popular choice for both small businesses and large enterprises.
“Secrets Manager” 220 is a service provided by AWS that helps one securely store, manage, and retrieve sensitive information such as passwords, API keys, and other credentials. It enables one to centralize and control access to secrets across AWS infrastructure and applications. With Secrets Manager 220, one can rotate, audit, and manage access to secrets programmatically, making it easier to adhere to security best practices and compliance requirements.
“Lambda” 230 is a serverless computing service that allows one to run code without provisioning or managing servers. One can upload code and Lambda 230 takes care of automatically scaling and managing the underlying infrastructure needed to run the code in response to incoming requests or events. Lambda 230 supports multiple programming languages, including Node.js, Python, Java, and more, making it versatile for various use cases such as data processing, backend services, automation, and event-driven applications.
“Cognito” 240 is a managed identity service that provides authentication, authorization, and user management for web and mobile applications. It allows one to easily add user sign-up and sign-in, as well as access control features to one's applications. With Cognito 240, one can authenticate users through social identity providers like Facebook and Google, enterprise identity providers like Active Directory, or a custom authentication system. Additionally, Cognito 240 offers features such as multi-factor authentication, user pools, and identity federation, making it a comprehensive solution for managing user identities in the provided applications.
“DynamoDB” 250 is a fully managed NoSQL database service that provides fast and predictable performance with seamless scalability. It is designed to handle large-scale, high-traffic applications that require low-latency access to data. DynamoDB 250 offers features such as automatic scaling, built-in security, backup and restore capabilities, and flexible data models. It supports both document and key-value data models, making it suitable for a wide range of use cases including web and mobile applications, gaming, IoT, and more. DynamoDB 250 is known for its simplicity, performance, and scalability, making it a popular choice for developers building modern, cloud-native applications.
“AppSync” 260 is a managed service that simplifies the process of building scalable applications with real-time and offline capabilities. It allows one to create GraphQL APIs to securely access and combine data from multiple sources, including AWS services like DynamoDB 250, Lambda 230, and more. With AppSync 260, one can easily build interactive and collaborative applications with features like real-time updates, offline data synchronization, and fine-grained access control.
“API Gateway” 270 is a fully managed service that makes it easy for developers to create, publish, maintain, monitor, and secure APIs at any scale. It acts as a front door for applications to access data, business logic, or functionality from backend services, like Lambda functions or EC2 instances, securely and at scale. With API Gateway 270, one can handle all aspects of the API lifecycle, including versioning, authentication, authorization, throttling, and monitoring, without having to manage the underlying infrastructure.
“Tools & SDKs” 280 refer to a comprehensive set of software development kits (SDKs), command-line tools, and other utilities provided by Amazon to facilitate building applications and managing AWS resources programmatically. These tools and SDKs are available in various programming languages, such as Python, Java, JavaScript, .NET, and more. They allow developers to interact with AWS services, automate tasks, deploy applications, and integrate AWS functionality into their software projects efficiently.

Also shown in FIG. 2 is “Convert API” 290, which is a third-party service that provides a suite of file conversion tools accessible via an API. It allows developers to integrate document, image, audio, and video conversion capabilities into their applications without needing to build these features from scratch. Convert API 290 supports a wide range of file formats and provides options for converting between them programmatically. Developers can leverage Convert API to add file conversion functionality to their applications, making it easier to handle various types of content.

The provided cloud-native application allows a user 310 to upload a listing and generate using a large language model 300 (e.g., ChatGPT) a corrective action plan. A computer interface may be provided to permit the editing of the generated corrective action plan. The computer interface also may permit the printing of a generated or edited corrective action plan. The computer interface also may permit the saving to memory of a generated or edited corrective action plan. The computer interface may also classify a generated or edited corrective action plan as being a work in progress (i.e., in review) or a work in final form.

In accordance with the system and method described herein, a method of creating a corrective action plan responding to a listing of corrective actions required includes the step of providing in a serverless hosting environment an application that: receives a hypertext transfer protocol request for a website to generate the corrective action plan; provides the website including functionality for uploading the listing of corrective actions required; stores the listing of corrective actions required in a data listing; provides the listing of corrective actions required to a large language model; requests that the large language model provide the corrective action plan based upon the listing of corrective actions required; receives the corrective action plan from the large language model; and stores the corrective action plan in the data listing. The website permits access to the corrective action plan. The corrective action plan can be edited via the website to create a modified corrective action plan. The modified corrective action plan can be stored in the data listing. A designation may be provided to a corrective action plan or a modified corrective action plan to indicate status. In one embodiment, the designation is selected from a group of designations including one or more of the following: new, under review, under final review, review complete, and final.

### Technical Advantages and Non-Generic Computer Processing

It should be understood that the description herein provides a technical improvement in computer/server performance and efficiency. What is described is not merely “authoring a corrective action plan on a computer.” Rather, the disclosed system and method changes how computers ingest, structure, and transform unstructured regulatory documents into machine-actionable data and then computationally assembles a compliant corrective action plan with fewer I/O operations, fewer repeated parsing cycles, and reduced user interactions. The improvement is realized at the systems level:
Layout-aware ingestion and normalization reduce repeated heavy parsing. Regulatory citations arrive as PDFs (including scans). OCR+layout segmentation is used to convert these into a normalized, tokenized representation once, then persist the result as structured JSON conforming to a schema. Subsequent requests operate on the structured representation rather than re-opening and re-parsing the PDF, reducing CPU cycles and disk I/O.
Standards mapping eliminates ad hoc searching. Each extracted deficiency is mapped to a canonical rule identifier (via XML/JSON rule maps). Because the data is pre-indexed to rule IDs, downstream components retrieve relevant constraints in O(1)/indexed lookups instead of broad text search, improving query-time efficiency and latency versus generic full-text operations.
Deterministic plan assembly engine reduces recomputation. The system binds facility-specific metadata, rule constraints, and prior fixes into a deterministic generation pipeline (state machine) that composes the corrective plan. Outputs are cached by content hash and rule ID, enabling deduplication and re-use across similar deficiencies, which lowers compute and network load in multi-user, multi-facility deployments.
Validation-first workflow lowers interaction and roundtrips. The system performs automated completeness checks (e.g., required elements per regulation, due dates, responsible parties, monitoring steps). Catching omissions at generation time avoids multi-step human back-and-forth and server roundtrips typical of generic editing tools, improving overall throughput.
Separation of heavy and light workloads for scalability. Heavy OCR/layout parsing runs in a background microservice; light, frequent operations (retrieval, rule checks, templated assembly) run in a low-latency service backed by indexes. This tiered design increases concurrency and system scalability without linear growth in compute cost.

In short, the system and method improves the functioning of the computer system itself by turning unstructured regulatory text into a schema-normalized, indexed corpus that the server can process with fewer expensive operations, while enabling deterministic, cacheable plan assembly.

Moreover, technical steps distinguish the system and method from generic computer operations. Each step in the step list below is technical (data transformation, indexing, stateful orchestration), not business logic:
1. Document Ingestion & OCR. Receive regulatory citations as PDFs (including scanned images). Apply OCR and layout segmentation (block, line, table detection) to produce a normalized text+structure graph.
2. Entity Extraction & Schema Normalization. Run extraction models/rules to identify deficiency tags, regulation numbers, dates, facility identifiers, and required remedial elements. Normalize results into a defined JSON schema (or XML) with fields for deficiency type, scope/severity, time frames, and required plan components.
3. Canonical Rule Mapping (Indexing). Map extracted deficiencies to canonical rule IDs in a maintained standards library (state-and program-specific). Persist rule-linked records so later requests hit indexed lookups rather than free-text search.
4. Constraint & Template Resolution. Retrieve rule-specific constraints (what must be addressed) and parameterized text blocks bound to the schema fields. Resolve facility metadata (e.g., policies, staffing roles) to fill variables.
5. Deterministic Plan Assembly Engine. Execute a state machine that composes the corrective action plan from validated components (root cause, corrective steps, responsible party, monitoring, completion dates). Enforce ordering and required elements; fail closed on missing data with machine-readable error codes.
6. Automated Completeness & Consistency Checks. Validate the assembled plan against rule constraints (e.g., all mandatory sections present; timelines realistic; monitoring measurable). Highlight conflicts/inconsistencies for user review within the UI (human-in-the-loop), prior to persistence.
7. Caching, Deduplication, and Reuse. Store assembled outputs and intermediate artifacts using content hashes and rule IDs. On similar future deficiencies, reuse validated components to avoid re-parsing and re-assembly.
8. Audit Trail & Versioned Storage. Persist versions and diffs as structured records (not just document blobs), enabling precise rollback and incremental updates without reprocessing the full document.
9. Low-latency Retrieval & Export. Serve the final plan from the structured store with indexed retrieval, and export to human-readable formats (PDF/Word) without recomputing the plan logic.

These steps collectively demonstrate specific, unconventional computer processing—OCR/layout analysis, schema normalization, canonical rule mapping, deterministic assembly, validation gates, and cache-based reuse—that go beyond generic “displaying/editing text” or “storing documents.

It should be understood that the foregoing description is only illustrative of the invention. Various alternatives and modifications can be devised by those skilled in the art having the benefit of this disclosure, without departing from the invention. Accordingly, the invention is intended to embrace all such alternatives, modifications and variances.

Certain exemplary embodiments of the disclosure may be described. Of course, the embodiments may be modified in form and content, and are not exhaustive, i.e., additional aspects of the disclosure, as well as additional embodiments, will be understood and may be set forth in view of the description herein. Further, while the invention may be susceptible to various modifications and alternative forms, specific embodiments have been shown by way of examples and described in detail herein. However, it should be understood that the invention is not intended to be limited to the particular forms disclosed. Rather, the invention is to cover all modifications, equivalents and alternatives falling within the spirit and scope of the invention.

What is claimed is:

1. A server computing system for providing corrective action plan information to a user is provided, the server computing system comprising:
a database storing corrective-action-related information for a healthcare facility;
a non-transitory computer readable storage device configured to store software instructions;
a computer processor configured to execute the software instructions to:
serve a website or mobile application content including user interface content renderable on a user computing device, wherein the user interface content comprises dynamic user interface controls for:
receiving information for the healthcare facility; and
presenting for the healthcare facility corrective-action-related information;
communicate the website or mobile application content to the user computing device, wherein the user computing device renders the website or mobile application content to display the user interface content;
receive the corrective-action-related information for the healthcare facility, wherein the corrective-action-related information is inputted via the dynamic user interface controls on the user computing device and the information is stored in the database as a structured representation;
access from the structured representation a recital of corrective action requirements;
serve an updated website or mobile application content including updated user interface content including a corrective action plan generated at least in part by a large language model operating on the recital of corrective action requirements; and
communicate the updated website or mobile application content to the user computing device, wherein the updated website or mobile application content is renderable by the user computing device to display the updated user interface content.

2. The system of claim 1, wherein accessing the recital comprises applying optical character recognition and layout segmentation to a regulatory PDF to produce a normalized text-and-structure representation.

3. The system of claim 1, wherein the corrective-action-related information is stored as a JSON or XML record conforming to a predefined schema including fields for regulation identifier, scope/severity, timelines, responsible roles, and monitoring steps.

4. The system of claim 1, further comprising mapping extracted deficiencies to canonical rule identifiers maintained in a standards library, thereby enabling indexed retrieval of rule-specific constraints.

5. The system of claim 1, wherein the corrective action plan is produced by a deterministic state-machine pipeline that composes required sections in a prescribed order and fails closed on missing required elements with machine-readable error codes.

6. The system of claim 1, further comprising automated completeness and consistency checks against rule-specific constraints prior to persistence of the corrective action plan.

7. The system of claim 1, wherein intermediate artifacts and outputs are content-hash indexed to enable deduplication and reuse across similar deficiencies.

8. The system of claim 1, wherein versions of the corrective action plan are stored as structured diffs enabling rollback and incremental updates without reprocessing the source document.

9. The system of claim 1, wherein finalized corrective action plans are served from indexed storage and exported to human-readable formats without recomputing plan logic.
